(19) United States
(12) Patent Application Publication
(10) Pub. No.: US 2005/0084510 A1
(43) Pub. Date: Apr. 21, 2005

(54) Water in oil formulations, method to prepare same, and personal care products formed using same

(76) Inventor: John C. Carson, Union City, NJ (US)

Correspondence Address: Dale F. Regelman;Law Office of Dale F. Regelman, P.C., 4231 S. Fremont Avenue, Tucson, AZ, 85714, US

(21) Appl. No.: 10/690,327

(22) Filed: Oct. 20, 2003

(51) Int. Cl. A61K 7/00

(52) U.S. Cl. 424/401

(57) ABSTRACT

A stable suspension formed of a plurality of water particles each of which is encapsulated with an amorphous silica-based material, where that plurality of encapsulated water particles is dispersed in a continuous phase formed of one or more carbon-containing materials, where the stable suspension does not include added emulsifiers. A method to prepare such stable suspensions. Personal care products comprising Applicant's stable suspensions, including skin protectants, sunscreens, moisturizers, vehicles for medicaments, antiperspirants, deodorants, pressurized products such as aerosol products, vehicles for skin treatment products and vehicles for makeup, area of the eye, lip products, mascara and color cosmetic products.

### FIELD OF THE INVENTION

The present invention relates to a stable suspension formed from amorphous silica encapsulated water particles dispersed in a carbon-containing continuous phase.

### BACKGROUND OF THE INVENTION

Suspensions of water, or various aqueous solutions, in air are known in the art. These suspensions comprise free-flowing powders that are cool to the touch. When compressed or rubbed between two surfaces, such as between the hands or fingers, these materials release their internal water. Initially, the released water appears as a liquid that has separated from the powder and which does not readily wet the surface. Upon continued rubbing, however, the released water combines with the residual powder material to form an aesthetically pleasing, paste-like mass that is pleasant to the touch, cooling to the skin and can be easily spread.

Conceptually, these internal water phase, dry powders can be thought of as an emulsion or dispersion of water in air with the powder acting as the emulsifier that prevents the close approach and agglomeration of the internal (water) phase. Such powder type water-in air products are commercially available.

While these “water in air” materials are interesting products that might seem to have strong consumer appeal, the technical difficulties associated with their storage and effective delivery to the skin, have prevented wide-spread commercial usage. Product stability for long term storage and shipping is, in one aspect, a function of the partial pressure of the water phase that is dispersed in the external, i.e. powder, phase. As the storage temperature increases, more water is volatilized from the internal phase and collects as vapor in the external powder phase. When the temperature is reduced, then the water vapor condenses into liquid water droplets that are not sufficiently stabilized by the external powder phase and thus precipitate and coalesce into a separated liquid water phase.

Further, the material of construction of the storage container may exacerbate this problem; for example glass containers are problematic. They seem to have a surface that is sufficiently hydrophilic such that either the internal phase is attracted to it or water vapor from the internal phase can collect and condense upon it, thus promoting separation.

To ameliorate these difficulties attendant with use of such commercial “water powders,” what is needed is a composition comprising a stable dispersion of such encapsulated water particles which facilitates storage and usage of those powders. Applicant's composition provides such a stable dispersion.

### SUMMARY OF THE INVENTION

Applicants' invention includes a suspension formed of a plurality of water particles each of which is encapsulated with an amorphous silica-based material, where that plurality of encapsulated water particles is dispersed in a continuous phase formed of one or more carbon-containing materials, where the suspension does not include added emulsifiers.

Applicant's invention further includes a method to prepare Applicant's suspensions. Applicant's invention further includes personal care products comprising Applicant's stable suspensions, including skin protectants, sunscreens, moisturizers, vehicles for medicaments, antiperspirants, deodorants, pressurized products such as aerosol products, vehicles for skin treatment products and vehicles for makeup, area of the eye, lip products, mascara and color cosmetic products.

### BRIEF DESCRIPTION OF THE DRAWINGS

The invention will be better understood from a reading of the following detailed description taken in conjunction with the drawings in which like reference designators are used to designate like elements, and in which:

FIG. 1A is a perspective view of Applicant's suspension;

FIG. 1B is a cross-sectional view of an encapsulated water particle;

FIG. 2 is a perspective view of prior art suspensions;

FIG. 3 is a table of formulations used to prepare Applicant's amorphous silica-based encapsulant;

FIG. 4A is a table of formulations used to prepare Applicant's encapsulated water particles;

FIG. 4B is a table of additional formulations used to prepare Applicant's encapsulated water particles;

FIG. 5 is a table of formulations used to prepare Applicant's stable suspensions;

FIG. 6 is a table of additional formulations used to prepare Applicant's stable suspensions; and

FIG. 7 is a flowchart summarizing Applicant's method to prepare his stable suspensions.

### DETAILED DESCRIPTION OF THE PREFERRED EMBODIMENTS

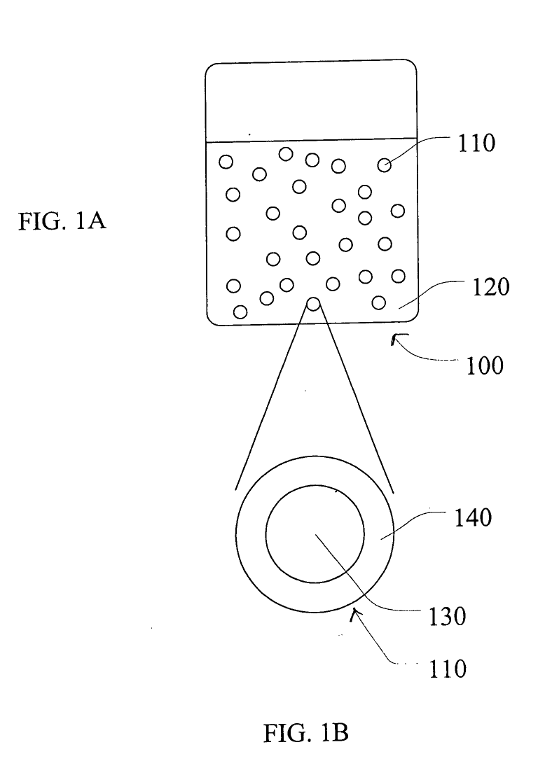

Referring to FIG. 1A, Applicant's invention includes stable dispersion 100 which comprises a plurality of individual particles 110 dispersed in continuous phase 120. The relative sizes of particles 110 has been increased in FIG. 1A for illustrative purposes. In certain embodiments, the plurality of particles 110 comprises a discontinuous, solid phase. By solid phase, Applicant means a material having both a fixed volume and a fixed shape at room temperature. In certain embodiments, continuous phase 120 comprises a liquid material. By liquid, Applicant means a material having a fixed volume, but a variable shape, at room temperature.

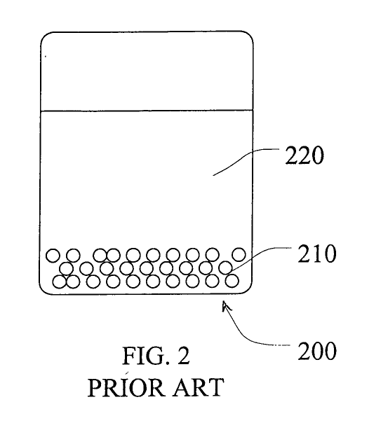

By forming Applicant's composition using Applicant's method, a stable dispersion of particles 110 can be formed in continuous phase 120. By stable dispersion, Applicant means a dispersion that is kinetically stable for at least 30 days at room temperature. By kinetically stable, Applicant means the absence of one or more processes, such as aggregation, through which dispersed phase 110 coalesces into a visually distinct component. FIG. 2 shows mixture 200 which comprises an unstable dispersion which has separated into visually distinct components. Mixture 200 represents many prior art dispersions which, without the use of one or more added stabilizers and/or emulsifiers, separates on standing into visually distinct, immiscible components, including first component 210 and second component 220.

Referring now to FIG. 1B, in certain embodiments of Applicant's invention the dispersed phase of Applicant's composition comprises what has sometimes been called a “water-in-air dispersion,” wherein a plurality of individual particles 110 each comprise an individual aqueous-based droplet 130 encapsulated by a solid material 140.

In certain embodiments, Applicant's aqueous-based component 130 includes additives that perform a variety of functions to enhance product stability, improve aesthetics, change the physical properties or modify the function of the product when applied to skin or hair. Such additives include, for example, antimicrobial agents, chelating agents, antioxidants, humectants, proteins, vitamins, medicaments, preservatives, polymers, hydrophilic clays, botanical extracts, colorants, pigments, fragrances, flavors, sweeteners and surfactants. Such additives are present from about 0 weight percent to about 20 weight percent of component 130.

Applicant's humectants include propylene glycol, sorbitol, maltitol, polydextrose, and the like. Applicant's chelating agents include ethylene diamine tetraacetic acid (EDTA), polyethyleneimine, poly-2-ethyl-2-oxazoline, citric acid, salts of citric acid, and the like.

In addition, Applicant has discovered that addition of salts to component 130 improves the separation, dispersion, and stability of the individual particles 110, and also improves the kinetic stability of dispersion 100. In certain embodiments of Applicant's invention component 130 includes salts having, for example, the formula M⁺X⁻ and/or M⁺⁺X⁻, M⁺⁺2X⁻, M⁺⁺⁺3X⁻, wherein the metal cation is selected from the group consisting of an alkali and/or alkali earth metal ion, Al⁺⁺⁺, Zn⁺⁺, Zr⁺⁴, and mixtures thereof, and wherein X⁻/X⁻⁻ is selected from the group consisting of chloride, fluoride, iodide, acetate, benzoate, sulfate, and the like. In these embodiments, such salt additives are present from about 0 weight percent to about 20 weight percent of component 130.

Encapsulant 140 comprises between about 3 weight percent to about 15 weight percent of particle 110. Aqueous-based liquid 130, including additives, comprises between about 85 weight percent and about 97 weight percent of particle 110. In certain embodiments, encapsulant 140 includes one or more additives such as talcs, clays, pigments, TiO₂, ZnO, polymer powders, powdered physiologically active materials, powdered antimicrobial agents and microencapsulated materials such as fragrances, oils, and/or emollients.

In certain embodiments, encapsulant 140 comprises a plurality of individual particles of amorphous silica. Such amorphous silica particles can be formed by the continuous flame hydrolysis of silicon tetrachloride SiCl₄. During high-temperature hydrolysis, gaseous SiCl₄ reacts with intermediately formed water in an oxy-hydrogen flame. The end result is a plurality of amorphous silica particles. By controlling the combustion conditions, it is possible to influence particle size distribution, surface area and surface properties.

In certain embodiments, encapsulant 140 is formed by treating amorphous silica formed as described above with chlorosilanes, such as (CH₃)₃—Si—Cl, which react with OH—Si groups on the surface of the silica particles to form a surface modified with (CH₃)₃—Si—O— moieties that are extremely hydrophobic. In these embodiments, this hydrophobic silica comprises encapsulant 130.

In certain embodiments of Applicant's invention, component 120 comprises a carbon-containing material. These embodiments of Applicant's composition comprise, in essence, a modified a water-in-oil emulsion which has enhanced stability compared with traditional water-in-oil emulsions, even without the use of one or more added surfactants and/or stabilizers.

The carbon-containing liquid component of Applicant's composition includes, for example, one or more hydrocarbons, including alkanes, alkenes, aromatic compounds, and mixtures thereof; triglycerides; esters; fatty alcohols, wherein the descriptor “fatty” means one or more carbon-based compounds having ten or more carbon atoms; fatty aldehydes; fatty ketones; fatty organic acids; fatty amines; dimethicones having structure I: 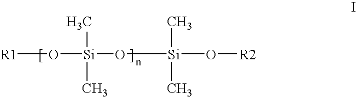
wherein R1 and R2 are selected from the group consisting of alkanes, alkenes, alkynes, aromatics, and mixtures thereof, and wherein R1 and R2 may be the same or may differ, and wherein n varies from about 2 to greater than 100; cyclomethicones, having structure II: 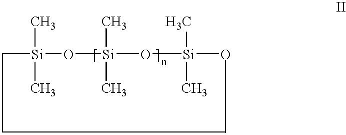
wherein n is between 2 and 6; perfluorinated materials; sunscreens; waxes; oils; fats; waxes; petrolatum; lanolin; and combinations of these materials. In certain embodiments, carbon-containing material 120 has a dielectric constant less than about 3.5.

In certain embodiments, carbon-containing component 120 comprises jojoba oil. Jojoba oil is obtained from the seed of the shrub Simmondsia chinensis which is native to the Sonoran desert. Jojoba oil is a mixture of naturally-occurring compounds obtained from the jojoba seed, sometimes called the jojoba bean. Jojoba seed contains about 50 weight percent of a yellow oil commonly referred to as jojoba oil. In contrast to other vegetable oils which comprise a mixture of triglycerides, jojoba oil comprises a mixture of long-chain esters.

As those skilled in the art will appreciate, carboxylic ester V can be formed by the reaction of alcohol IV and carboxylic acid III. In addition, an ester-group-containing compound, such as many of the constituents of jojoba oil, can be described as comprising an R5 component and an R6 component.

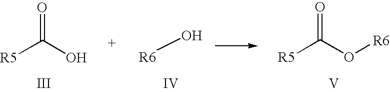

Jojoba oil includes a variety of ester-group-containing compounds wherein the R5 component comprises a mixture of carbon-containing moieties having, primarily, 17, 18, 20, and 22 carbon atoms, and wherein the R6 component comprises a mixture of carbon-containing moieties having, primarily, 18, 20, 22, and 24 carbon atoms. Furthermore, it is known that the R5 component of these various jojoba oil ester-group-containing constituents includes at least one carbon-carbon double bond having a cis-configuration. Sometimes such a cis-configuration is known as the Z-configuration. It is further known that the R6 component of these various jojoba oil ester-group-containing constituents includes at least one carbon-carbon double bond having a cis-configuration. Sometimes such a cis- configuration is known as the Z-configuration.

Certain derivatives of jojoba oil are known in the art. For example, isomerization of the double bond in the R5 component, and/or the R6 component, of the various jojoba esters from the cis configuration to a trans configuration yields a material that is solid at room temperature, where that solid material includes one or more crystalline compounds. U.S. Pat. No. 4,329,298 teaches a method to isomerize jojoba oil and is hereby incorporated herein by reference.

In addition, hydrogenation of the double bond in the R5 component, and/or hydrogenation of the double bond in the R6 component, of the jojoba oil ester yields a crystalline, wax-like material. Substantially fully hydrogenated jojoba oil is a solid with a melting point upwards of 70° C. As those skilled in the art will appreciate, the degree of hydrogenation can be measured using an Iodine Value (“IV”). Naturally-occurring jojoba oil has an IV of between about 80 and 85. As the percentage of carbon-carbon double bonds hydrogenated increases, the IV of that hydrogenated material decreases. As the percentage of carbon-carbon double bonds hydrogenated increases, the degree of crystallinity and the melting point of that hydrogenated material also increase.

Those skilled in the art will further appreciate that the viscosity of mixtures of jojoba oil and one or more jojoba oil derivatives is determined by the kind and amounts of the various ester compounds comprising that oil. Certain mixtures of jojoba oil and jojoba oil derivatives are liquid at room temperature. Certain mixtures of jojoba oil and jojoba oil derivatives are solid at room temperature. Certain mixtures of jojoba oil and jojoba oil derivatives are semi-solid at room temperature. By “semi-solid,” Applicant means a material that comprises one or more liquids in combination with one or more solids.

Applicant's invention includes personal care products comprising Applicant's stable suspension. Such personal care products include skin protectants, sunscreens, moisturizers, vehicles for medicaments, antiperspirants, deodorants, pressurized products such as aerosol products, vehicles for skin treatment products and vehicles for makeup, area of the eye, lip products, mascara and color cosmetic products

The following examples are presented to further illustrate to persons skilled in the art how to make and use the invention and to identify presently preferred embodiments thereof. These examples are not intended as limitations, however, upon the scope of the invention, which is defined only by the appended claims.

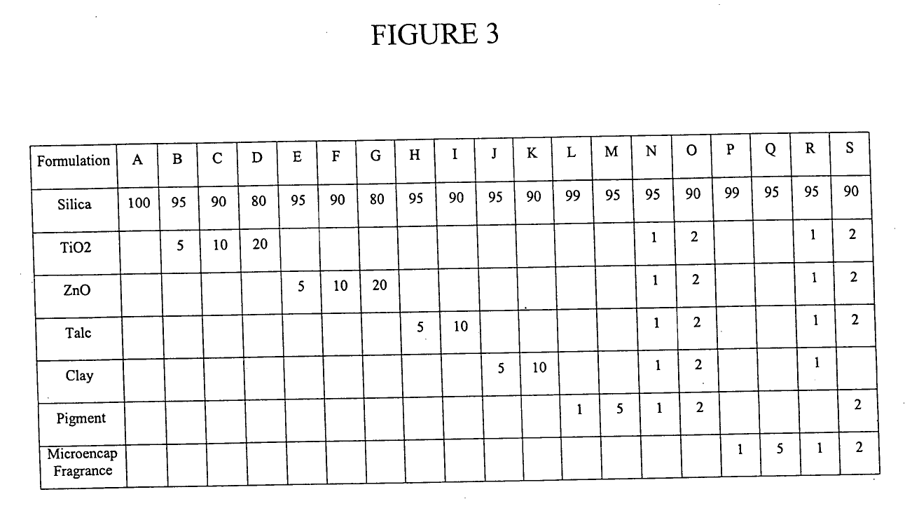

Furthermore, attention is directed to FIGS. 3, 4A, 4B, 5, and 6 which recite a more complete recitation of the formulations comprising Applicant's invention. All amounts recited in Examples I, II, III, and IV, represent weight percentages.

### EXAMPLE I

| | |
|---|---|
| Isomerized Jojoba Oil | 50.0 |
| Fumed Silica (Aerosil 972) | 5.0 |
| Water | 45.0 |

Aerosil® R-972 is sold in commerce by the Degussa Corporation. The Isomerized Jojoba Oil of Example I is sold in commerce by the assignee of this Application, namely Desert Whale Jojoba Company, Tucson, Ariz., under the product name Iso-Jojoba, and has a melting point of about 37° C. The product of Example I is a soft solid that spreads easily and releases the internal water phase as the Isomerized Jojoba Oil melts with rubbing on the skin.

### EXAMPLE II

| | |
|---|---|
| Isomerized Jojoba Oil | 25.0 |
| Cyclomethicone (D-5) | 25.0 |
| PEG 120 Jojoba Wax | 5.0 |
| Fumed Silica (Aerosil 972) | 4.5 |
| Water | 40.5 |

Cyclomethicone (D-5) is sold in commerce by Dow Corning Corp. PEG 120 Jojoba Wax is sold in commerce by Floratech, 1151 N. Fiesta Blvd, Gilbert, Ariz. The Isomerized Jojoba Oil of Example II has a melting point greater than about 37° C. The product of Example II is a harder formulation that is suitable for casting in stick form which can used in, for example, lip gloss and/or lip stick formulations.

### EXAMPLE III

| | |
|---|---|
| Aluminum Sesquichlorohydrate | 45.0 |
| (50% aqueous solution) | |
| Fumed Silica (Aerosil 972) | 5.0 |
| Cyclomethicone (D-5) | 50.0 |

The product of Example III is a viscous, but pourable, opaque, white emulsion that allows the release of the antiperspirant ingredient. The product is suitable for dispensing as a cream from a propel type cream/gel dispenser, from pad applicators or from roller ball type applicators. The product of Example III is useful as an antiperspirant.

### EXAMPLE IV

| | |
|---|---|
| Aluminum Sesquichlorohydrate | 45.0 |
| (50% aqueous solution) | |
| Fumed Silica (Aerosil 972) | 5.0 |
| Cyclomethicone (D-5) | 22.3 |
| Isomerized Jojoba Oil | 25.0 |
| Arlacel 165 | 2.5 |
| Fragrance | 0.2 |

Arlacel 165 is a surfactant sold in commerce by ICI Atkemix, Brantford, Ontario, Canada N3T 5T2. The product of Example IV is a high viscosity, opaque, white paste that applies smoothly with the fingers or from a propel type cream/gel dispenser leaving no visible residue on the skin. The product of Example IV is useful as an antiperspirant.

Applicant's invention includes a method to prepare the compositions discussed above. First, the water-in-air dispersion comprising plurality of particles 110 is formed. Thereafter, the component 120, i.e. the continuous phase of suspension 100, is added either directly into the high speed disperser used to form particles 110, or alternatively into another mixer such as a high speed propeller or turbine mixer. Applicant has found, however, that attempts to prepare Applicant's suspension by simultaneously combining aqueous-based component 130, encapsulant 140, and carbon-containing material 120 in a high speed disperser mixer yields a mass of component 120 gelled by component 140 surrounded by an external water phase—not the desired product.

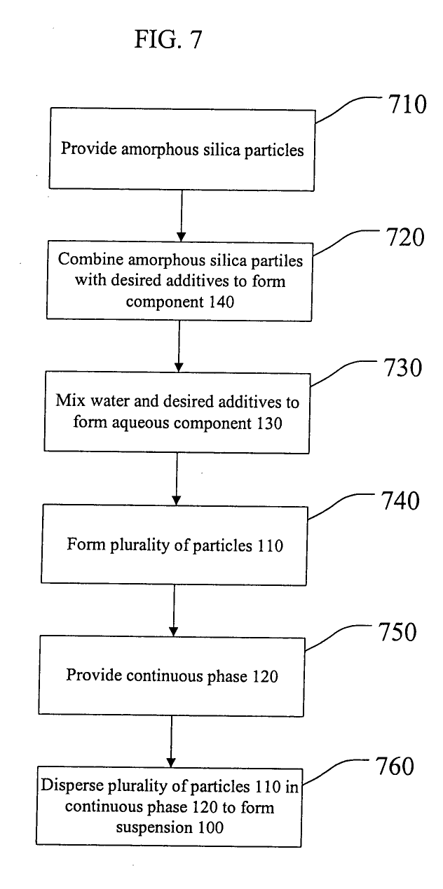

FIG. 7 summarizes the steps of Applicant's method. In step 710, amorphous silica particles are provided. In certain embodiments, step 710 comprises the flame combustion process discussed above to form amorphous SiO₂ particles from SiCl₄ using an oxy-hydrogen flame. In certain embodiments, step 710 further includes reacting the plurality of amorphous silica particles with one or more chlorosilanes having the formula R₁R₂R₃Si—Cl to form hydrophobic amorphous silica particles, wherein R₁, R₂, and R₃, are alkyl, such as for example methyl, ethyl, propyl, and the like, and wherein R₁, R₂, and R_{3 may be the same or may differ.}

In step 720, the amorphous silica particles are combined with desired additives, such as TiO₂, ZnO, talcs, clays, and/or microencapsulated fragrances, to form component 140 (FIG. 1B) which comprises one of Formulations A through S (FIG. 3). In step 730, aqueous-based component 130 is prepared by mixing water and any desired additives, such as antimicrobial agents, chelating agents, antioxidants, humectants, proteins, vitamins, medicaments, preservatives, polymers, hydrophilic clays, botanical extracts, colorants, pigments, fragrances, flavors, sweeteners and surfactants.

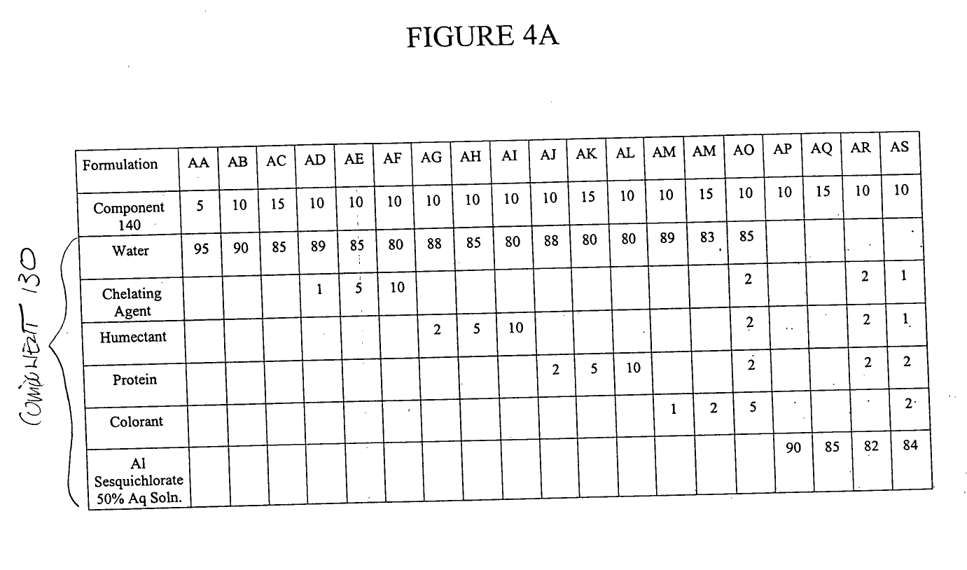

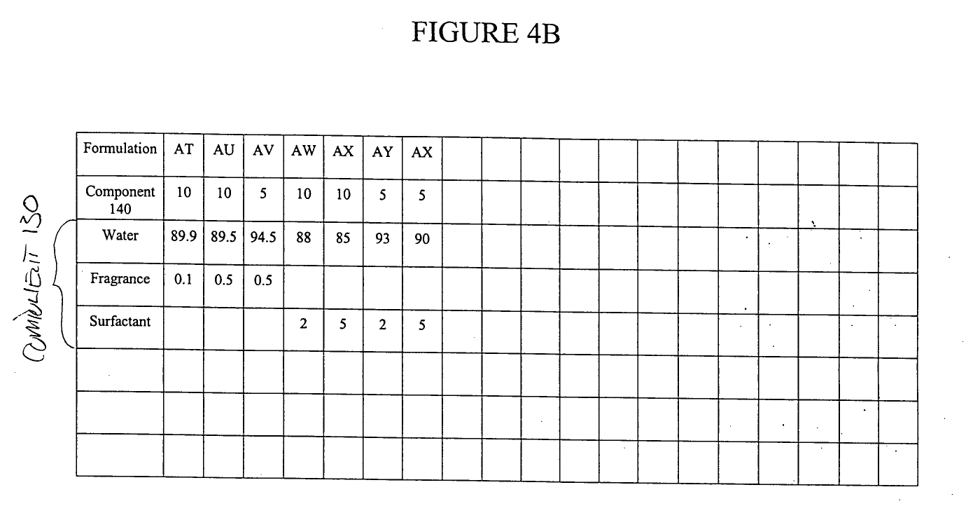

In step 740, aqueous-based component 130 is dispersed into a plurality of water particles in the presence of a plurality of encapsulant particles 140 to form a plurality of particles 110. Each of the individual water particles becomes encapsulated by a portion of the plurality of encapsulant particles. FIGS. 4A and 4B recites the formula amounts of component 140, and the ingredients comprising component 130. In certain embodiments, the components 130 and 140 are added to a blender which is operated at maximum speed for about 30 seconds to about 120 seconds, or until the powder has become noticeably denser and more uniform. In embodiments wherein larger quantities of particles 110 are formed, i.e. production of commercial quantities, a mechanical fluidized bed mixing apparatus comprising a high sheer mixer can be used.

Over blending, i.e. mixing for too long a period of time, is to be avoided. Such over blending can cause the encapsulant 140 to wet thus forming a paste, i.e. a dispersion of the powder in the water phase, rather than the desired encapsulation of the aqueous-based component 130 in the encapsulant 140. Applicant has found that the dispersion of the aqueous phase in the powder phase must be done quickly to avoid wetting the powder and it must be done so as to produce a maximum of small sized, uniform water phase droplets in order to maximize the stability of the encapsulated water particles 110.

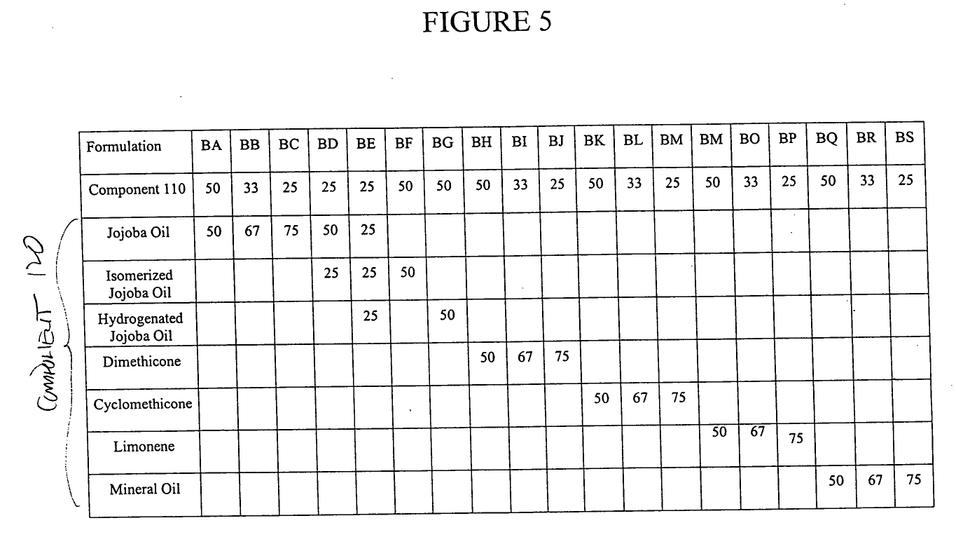

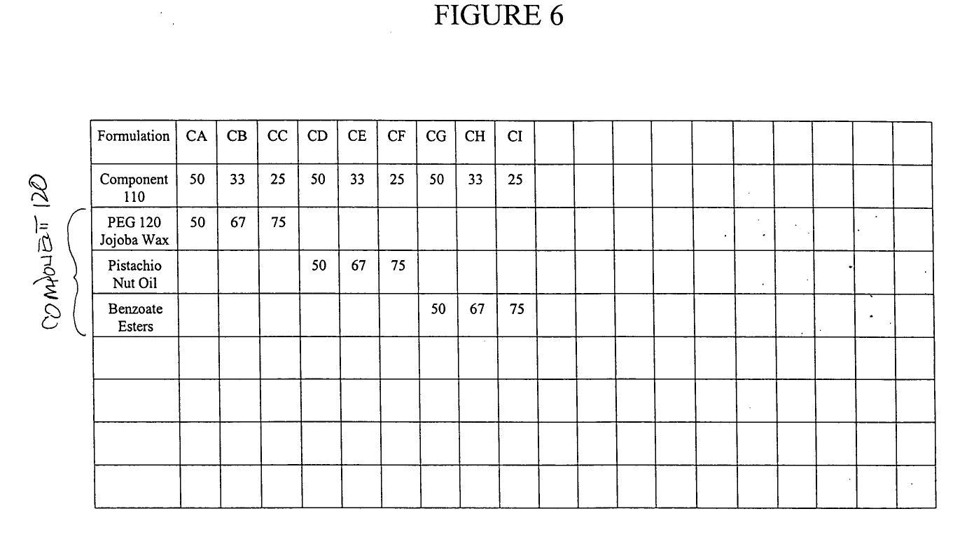

In step 750, component 120 is prepared. In certain embodiments, step 750 includes the step of heating a viscous carbon-containing material to a temperature sufficient to lower the viscosity to about 200 cps. In step 760, the plurality of particles 110 are dispersed in component 120 to form suspension 100 using a high sheer mixing apparatus. FIGS. 5, 6 recites formula amounts for component 110 and the ingredients comprising component 120. In certain embodiments, continuous phase 120 is added either directly into the high speed disperser used in step 740, or alternatively into another mixer such as a high speed propeller or turbine mixer.

While the preferred embodiments of the present invention have been illustrated in detail, it should be apparent that modifications and adaptations to those embodiments may occur to one skilled in the art without departing from the scope of the present invention as set forth in the following claims.

1. A method to form a stable suspension, comprising the following steps in the following order:
(a) providing a plurality of amorphous silica particles;
(b) mixing said plurality of amorphous silica particles with water;
(c) forming a plurality of amorphous silica encapsulated water particles;
(d) providing a carbon-containing material;
(e) dispersing said plurality of amorphous silica encapsulated water particles in said carbon-containing material to form said suspension.

2. The method of claim 1, wherein said method does not include the step of adding one or more emulsifying agents.

3. The method of claim 1, further comprising the step of combining said plurality of amorphous silica particles with one or more first additives after step (a), wherein said one or more first additives are selected from the group consisting of talcs, clays, pigments, TiO₂, ZnO, polymer powders, powdered physiologically active materials, powdered antimicrobial agents and microencapsulated materials such as fragrances, oils, and emollients.

4. The method of claim 1, further comprising the step of combining water with one or more second additives after step (a), wherein said one or more second additives are selected from the group consisting of antimicrobial agents, chelating agents, antioxidants, humectants, proteins, vitamins, medicaments, preservatives, polymers, hydrophilic clays, botanical extracts, colorants, pigments, fragrances, flavors, sweeteners, surfactants, and salts.

5. The method of claim 1, wherein said mixing step is performed for about 120 seconds or less.

6. The method of claim 1, wherein said carbon-containing material has a room temperature viscosity greater than about 200 cps, further comprising after step (d) the step of heating said carbon-containing material to lower its viscosity to about 200 cps.

7. The method of claim 1, wherein said carbon-containing material has a dielectric constant less than about 3.5.

8. The method of claim 1, wherein said carbon-containing material comprises one or more alkanes, alkenes, aromatic compounds, triglycerides, esters, fatty alcohols, fatty ketones, fatty acids, fatty amines, dimethicones, cyclomethicones, perfluorinated materials, sunscreens, waxes, oils, fats petrolatum, lanolin, pistachio nut oil, benzoate esters, limonene, and combinations thereof.

9. The method of claim 1, wherein said carbon-containing material comprises jojoba oil.

10. The method of claim 9, wherein said jojoba oil comprises isomerized jojoba oil.

11. The method of claim 9, wherein said jojoba oil comprises hydrogenated jojoba oil.

12. A suspension, comprising:
a plurality of water particles;
a plurality of amorphous silica particles;
wherein each of said plurality of water particles is encapsulated by a portion of said plurality of amorphous silica particles to form a plurality of silica encapsulated water particles;
a carbon-containing material;
wherein said plurality of encapsulated water particles are dispersed in said carbon-containing material to form said suspension.

13. The suspension of claim 12, wherein said plurality of amorphous silica particles comprise one or more first additives.

14. The suspension of claim 13, wherein said one or more first additives are selected from the group consisting of talcs, clays, pigments, TiO₂, ZnO, polymer powders, powdered physiologically active materials, powdered antimicrobial agents and microencapsulated materials such as fragrances, oils, and emollients.

15. The suspension of claim 12, wherein each of said plurality of water particles comprises one or more second additives.

16. The suspension of claim 15, wherein said one or more second additives are selected from the group consisting of antimicrobial agents, chelating agents, antioxidants, humectants, proteins, vitamins, medicaments, preservatives, polymers, hydrophilic clays, botanical extracts, colorants, pigments, fragrances, flavors, sweeteners, surfactants, and salts.

17. The suspension of claim 12, wherein said carbon-containing material has a dielectric constant less than about 3.5.

18. The suspension of claim 12, wherein said carbon-containing material comprises one or more alkanes, alkenes, aromatic compounds, triglycerides, esters, fatty alcohols, fatty ketones, fatty acids, fatty amines, dimethicones, cyclomethicones, perfluorinated materials, sunscreens, waxes, oils, fats petrolatum, lanolin, pistachio nut oil, benzoate esters, limonene, and combinations thereof.

19. The suspension of claim 12, wherein said carbon-containing material comprises jojoba oil.

20. The suspension of claim 19, wherein said jojoba oil comprises isomerized jojoba oil.

21. The suspension of claim 19, wherein said jojoba oil comprises hydrogenated jojoba oil.

22. A personal care product, comprising:
a plurality of water particles;
a plurality of amorphous silica particles;
wherein said each of said plurality of water particles is encapsulated by a portion of said plurality of amorphous silica particles to form a plurality of silica encapsulated water particles;
a carbon-containing material;
wherein said plurality of encapsulated water particles are dispersed in said carbon-containing material to form said suspension.

23. The personal care product of claim 22, wherein said plurality of amorphous silica particles comprise one or more first additives.

24. The personal care product of claim 23, wherein said one or more first additives are selected from the group consisting of talcs, clays, pigments, TiO₂, ZnO, polymer powders, powdered physiologically active materials, powdered antimicrobial agents and microencapsulated materials such as fragrances, oils, and emollients.

25. The personal care product of claim 22, wherein each of said plurality of water particles comprises one or more second additives.

26. The personal care product of claim 25, wherein said one or more second additives are selected from the group consisting of antimicrobial agents, chelating agents, antioxidants, humectants, proteins, vitamins, medicaments, preservatives, polymers, hydrophilic clays, botanical extracts, colorants, pigments, fragrances, flavors, sweeteners, surfactants, and salts.

27. The personal care product of claim 22, wherein said carbon-containing material has a dielectric constant less than about 3.5.

28. The personal care product of claim 22, wherein said carbon-containing material comprises one or more alkanes, alkenes, aromatic compounds, triglycerides, esters, fatty alcohols, fatty ketones, fatty acids, fatty amines, dimethicones, cyclomethicones, perfluorinated materials, sunscreens, waxes, oils, fats petrolatum, lanolin, pistachio nut oil, benzoate esters, limonene, and combinations thereof.

29. The personal care product of claim 22, wherein said carbon-containing material comprises jojoba oil.

30. The personal care product of claim 29, wherein said jojoba oil comprises isomerized jojoba oil.

31. The personal care product of claim 30, wherein said jojoba oil comprises hydrogenated jojoba oil.

32. A lip care product, comprising:
a plurality of water particles;
a plurality of amorphous silica particles;
wherein said each of said plurality of water particles is encapsulated by a portion of said plurality of amorphous silica particles to form a plurality of silica encapsulated water particles comprising about 90 weight percent water and about 10 weight percent amorphous silica;
isomerized jojoba oil;
wherein said plurality of encapsulated water particles are dispersed in said isomerized jojoba oil to form a suspension comprising said lip care product.

33. A lip care product, comprising:
a plurality of water particles;
a plurality of amorphous silica particles;
wherein said each of said plurality of water particles is encapsulated by a portion of said plurality of amorphous silica particles to form a plurality of silica encapsulated water particles comprising about 90 weight percent water and about 10 weight percent amorphous silica;
isomerized jojoba oil;
cyclomethicone;
PEG 120 jojoba wax;
wherein said plurality of encapsulated water particles are dispersed in a mixture comprising said isomerized jojoba oil, said cyclomethicone, and said PEG 120 jojoba wax, to form said lip care product.

34. An antiperspirant, comprising:
a plurality of water particles comprising a 50 weight percent aqueous solution of Aluminum Sesquichlorohydrate;
a plurality of amorphous silica particles;
wherein said each of said plurality of water particles is encapsulated by a portion of said plurality of amorphous silica particles to form a plurality of silica encapsulated water particles;
cyclomethicone;
wherein said plurality of encapsulated water particles are dispersed in said cyclomethicone to form said antiperspirant.

35. The antiperspirant of claim 34, wherein:
said 50 weight percent aqueous solution of Aluminum Sesquichlorohydrate is present in said antiperspirant at about 45 weight percent;
said amorphous silica particles are present in said antiperspirant at about 5 weight percent; and
said cyclomethicone is present in said antiperspirant at about 50 weight percent.

36. An antiperspirant, comprising:
a plurality of water particles comprising a 50 weight percent aqueous solution of Aluminum Sesquichlorohydrate;
a plurality of amorphous silica particles;
wherein each of said plurality of water particles is encapsulated by a portion of said plurality of amorphous silica particles to form a plurality of silica encapsulated water particles;
isomerized jojoba oil;
cyclomethicone;
a surfactant;
a fragrance;
wherein said plurality of encapsulated water particles are dispersed in a mixture comprising said isomerized jojoba oil, said cyclomethicone, said surfactant, and said fragrance, to form said antiperspirant.

37. The antiperspirant of claim 36, wherein:
said 50 weight percent aqueous solution of Aluminum Sesquichlorohydrate is present in said antiperspirant at about 45 weight percent
said amorphous silica particles are present in said antiperspirant at about 5 weight percent;
said cyclomethicone is present in said antiperspirant at about 22.3 weight percent;
said surfactant is present in said antiperspirant at about 2.5 weight percent; and
said fragrance is present in said antiperspirant at about 0.2 weight percent.
